(19) United States
(12) Patent Application Publication
(10) Pub. No.: US 2007/0043320 A1
(43) Pub. Date: Feb. 22, 2007

(54) Microstream injector

(76) Inventor: Saad Al Kenany, Palo Alto, CA (US)

Correspondence Address: GREENBERG TRAURIG, LLP, 1900 UNIVERSITY AVENUE, FIFTH FLOOR, EAST PALO ALTO, CA, 94303, US

(21) Appl. No.: 11/350,463

(22) Filed: Feb. 8, 2006

Related U.S. Application Data
(60) Provisional application No. 60/651,563, filed on Feb. 9, 2005.

(51) Int. Cl. A61M 5/30

(52) U.S. Cl. 604/68

(57) ABSTRACT

The present invention provides a micromachined or microsized component-based needleless injector for delivering a dose of a liquid formulation containing a biologically active agent to tissue by way of a high pressure liquid microstream that penetrates the skin and deposits the agent at an optimal depth in the tissue. The device is appropriate for subcutaneous, intramuscular, or mucosal injection sites, as well as intracellular injection. Embodiments include micromachined or microsized components such as valves, jets, and MEMS pumps. Some embodiments of the invention are modular, with interchangeable parts, other embodiments are integrated in a unitary design. Embodiments with a unitary design are typically single use, however modular features also create embodiments with components that provide multiple instances of use.

### RELATED APPLICATIONS

The present application claims priority to U.S. Provisional Patent Application No. 60/651,563, of Saad Al Kenany, filed on Feb. 9, 2005.

### FIELD OF THE INVENTION

This technology relates to needleless devices for the administration of therapeutic agents, and methods of their use.

### BACKGROUND

Under optimal conditions, conventional administration of injectable drugs is by way of a hypodermic or pneumatic syringe that is operated by trained, skilled professionals in facilities where the syringe is properly disposed of after use. Even under optimal conditions, however, the use of needle syringes puts health workers and patients at risk of infection through inadvertent needle-sticks or equipment misuse. Additionally, use of needles can be painful, even when applied properly, and can cause tissue damage, and induce anxiety in patients.

Further, in the real and not-optimal world, there are situations where injections are given to large numbers of individuals, over a short period of time, under conditions that make it difficult or cost prohibitive to ensure that the syringes will not be reused. Since syringes and needles are difficult to disinfect or sterilize this improper use greatly increases the risk of blood-borne disease transmission among injection recipients. Reuse typically occurs in settings outside the formal health care system where the administration of the composition is done by non-professional personnel. In fact, it is estimated that 30-50% of all injections in developing countries are not done in a sterile or antiseptic manner.

To alleviate the risk of disease transmission and to help prevent misuse of pneumatic syringes, needle syringes have been developed that are disposable. However, these disposable units, once disposed, create hazardous waste, and waste disposal problems, as inadvertent needle sticks and spread of infectious disease may result from the mishandling of such medical waste. Further in the real world, some syringes that are designed to be disposable are, in fact, reused without being properly sanitized. Conservative estimates suggest that in the year 2000, 260,000 new cases of HIV, 21 million Hepatitis B infections, and 2 million Hepatitis C infections all resulted from contaminated injections. Therefore, the mishandling and misuse of these devices leads to an increased risk of many blood borne diseases such as: hepatitis B, Tetanus toxoid, and HIV. Still further, it is estimated that there are over 1.4 million deaths per year that occur as a result of the reuse of hypodermic needles and syringes.

As further motivated by safety and convenience considerations, pre-filled, single dose injection devices have been developed. These devices are designed to preclude or prevent reuse and often combine the medicament with the syringe and needle in a single foil package. Some single use syringes are configured such that when the injection bladder is compressed it collapses and is thus rendered incapable of being refilled after its initial use. Other types of these devices have a locking mechanism that is actuated such that when the walls of the bladder portion are squeezed the inner surfaces interlock preventing reuse. Still others are configured in a way that when they are used the piston effectively destroys the syringe. There are, however, various problems inherent in such no-second use devices. First, they are expensive to manufacture and/or are bulky and thus inconvenient to ship and store. Second, they still do include a needle apparatus that brings with it the attendant problems described above. Additionally, these pre-filled, single dose injection devices require training to be used properly and safely, and thus are not well adapted for use by untrained medical workers. Finally, even in the hands of trained medical personnel, needlesticks are not precluded by use of these devices, as their use accounts for at least a quarter of reported needlestick incidents.

The skin of vertebrate animals, including that of humans, provides a substantial barrier to chemical permeation; the stratum corneum of the epidermis can be likened to a wall-like brick and mortar structure. The corneocytes of keratin comprise the bricks, while the lipid bilayers, fatty acids, cholesterol comprise the mortar. Additionally, the skin surface contains cellular debris, microorganisms, sebum, and other materials that can negatively affect permeation. For hypodermic or pneumatic syringe based administrations the stratum corneum is bypassed by the injection of the needle through the dermis. However, the injective process brings with it an increased amount of pain and anxiety in the recipient, as well as all the attendant problems set forth in the background. Patch-based injectors avoid the pain and anxiety evoked by traditional injectors, however, the stratum corneum presents a real problem for patch systems, as the stratum corneum impedes permeation into deeper levels of the dermis. Because the diffusion is determined by steady state flux only certain drug characteristics provide the correct physiochemical properties that will allow translocation of a drug or composition across the stratum corneum and its attendant barriers.

In spite of the various technological approaches to the administration of therapeutic agents, basic considerations associated with needle syringes, such as cost, convenience and simplicity of use, and safety very much remain challenges in the delivery of health care. There is a need for a needleless medicament injector that can more safely be used and disposed of by medical care workers, professionals and untrained workers alike, as well as self-treating patients.

### SUMMARY OF THE INVENTION

The present invention is a needless injector of biologically active agents that operates by way of high power, narrow-diameter fluid microstream jets. Embodiments of the invention include devices with a single jet, as well as devices with multiple jets configured into an array. These microstream jets eject from open tubes that range in diameter from about 0.25 to about 60μ in diameter, more particularly from about 0.5 to about 40μ in diameter, and still more particularly from about 1μ to about 20μ in diameter. “Microstream”, or “microstream jets”, as used herein, encompasses propulsed streams of fluid with diameters that range, in accordance with the diameter range of the ejecting tubes, i.e., between about 0.25μ and about 60μ, more particularly between about 0.5μ and about 40μ, and still more particularly between about 1μ and about 20μ.

The propulsive force of the jet varies in accordance with the intended target site, and the intended penetrative depth of injection at that target site. The advantage provided by the microstream jet is that biologically active agent is effectively conveyed to the desired site without the use of a needle. The propulsive power is sufficient to pierce the tough stratum corneum that protects skin; and the propulsive power can be modulated, in accordance with the diameter of the microstream and the number of parallel streams, in order to deliver biological agents to the desired depth below the injection site, within the underlying tissue.

This invention has utility in the injected delivery of agents to subcutaneous sites, submucosal sites, and going deeper into the body, agents can be delivered intramuscularly. Biologically active agents can also be injected internally, within the body, in which case the stratum corneum barrier provided by skin in not present. As such, for example, biologically agents could be delivered by an injector threaded through a catheter to vascular, cardiac, or other organ system sites. Typically, biologically active agents are pharmaceutical agents used in the treatment or prevention of disease. Other biologically active agents, as embodied by this invention, include vaccines, viruses, DNA, RNA, or other compounds used in genetic engineering or gene therapy procedures, where the target is cellular, in contrast to a tissue target, or a systemic treatment, where the site of injection is merely an entry point into the bodily system as a whole. As with subcutaneous or tissue injection, power and stream width, and stream number are configured to be sufficient to penetrate a living cell, and yet not destroy it.

Embodiments of invention provide for micro-machined components which as a whole represent a microfluidic transport system, including a valve apparatus, convergent nozzles, and microjet-emitting tubes. These components are typically manufactured by micro-electro-mechanical system (MEMS) based manufacturing methods, in the context of so-called MEMS Fabs (“Fab” refers to fabric, fabrication, fabricator, or more simply, a factory). Other methods and nuances of manufacture may be available, or evolve, or become available at some point in the future; what is significant for the purpose of this invention is not the specifics of the method of manufacture, but rather the rendering of microsize components, as provided, for example, by micromachine methods. In some embodiments of the invention, the micro-machined components provide a passive microfluidic flow path, where the propulsive force is generated distal to the flow path, and culminates in the microstream jet as it emits from the tubes and penetrates the targeted injection site. In other embodiments of the invention, the micromachined components, themselves, participate electrically in the generation of propulsive force. In such embodiments, the valve apparatus includes a MEMS pump, or a multiplicity of MEMS pumps. Such pumps, in various embodiments, may either provide all of the propulsive force, or a portion of it. Some embodiments of the invention include a propulsive module which supplies the remaining force required for propulsion of the microstreams by way of convenionional (non-MEMS mechanisms) provided by other conventional propulsive mechanisms and methods, such as are provided by the release of compressed gas, release of springs, or ignition of pyrotechnic systems. The power released by these propulsive events is captured and conveyed to the liquid formulation by a force conveyer interposed between the propulsive module and the holding reservoir, such that fluid is conveyed through the microfluid transport assembly, and ultimately ejected through the ejecting tubes. This force conveyer, in various embodiments of the invention may be an elastic membrane surrounding the reservoir, a diaphragm, or a piston.

Included in embodiments of the invention are micromachined plates or gates, which together function as a valve system that regulates microfluidic flow. These valves typically take the form of complementary plates aligned adjacently to each other, parallel in their broadest aspect; each gate includes holes for microfluidic flow. When the gates are aligned in such a way that the holes in the respective complementary gates are co-aligned, or in-register, a contiguous microfluidic flow path is created, and the gate or valve is thus “open”. In contrast, when the gates are aligned in a position such that the holes of each gate are met by a solid wall on the other gate, there is no flow path, and the gate is accordingly “closed”. Typically there are two such plates, but embodiments of the invention include multiple plates as well. Each of the two or more respective plates has holes within it, and the plates have two or more positions with respect to each other, at least one of them being a closed position. Some embodiments of the invention include gates that are disc-shaped and rotatable; the discs are aligned on a common axis, and a rotary motion of the discs with respect to each other adjusts the alignment of the holes in the discs into an “off position” and one or more “open” positions. In other embodiments, the gates are non-rotatable plates, with holes in each respective plate, and the movement is linear, i.e., with the gates moving sideways with respect to each other. In these embodiments of the invention, typically, one of the gates moves, and the other is stationary. It is not necessary, however that movement is restricted to one gate, and in some embodiments of the invention both gates move, or are configured to be able to move during the operation of the gates as a valve.

Embodiments of the invention include models with a single microjet emitting tube, as well as models with multiple tubes, configured into an array. These micromachined tubes may be formed as tubes imbedded in a matrix, or as tubes etched into a matrix. The convergent nozzles upstream from the emitting tubes may converge on a single emitting tube, or an array or cluster of tubes. Embodiments of the invention include devices with a single convergent nozzle, or with an array of nozzles. All components of the microfluidic transport system may be coated with compositions that are appropriate for the composition of the liquid being transported. Depending on the polarity of the fluid, polymers or varying degree of hydrophobicity or hydrophilicity may be included. In some embodiments, the coating may be metallic.

Upstream from the micromachined valve apparatus, and representing the starting point of the microfluidic flowpath, is a reservoir for holding the biologically active agent until the time of injection. Typically, the biologically active agent is dissolved in a liquid formulation or a gel formulation. In some cases, a biologically active agent is more stable over the long term in a dry or lyophilized form. To accommodate this, some embodiments of the invention have a reservoir with a compartment to hold the dry agent, and a separate compartment containing an appropriate diluent. As a step preliminary to injection, mechanisms within the reservoir or associated with it, cause the combining of the dry formulation with the diluent to create a liquid formulation. These embodiments include a window in the reservoir so that the combined agent and diluent can be visually inspected to insure that dissolution is complete. This preliminary step, according to embodiments of the invention, may be set in motion manually by the user, prior to using the injector, or, in other embodiments, such combination may be automatically effected by mechanisms set into motion by an alerting event initiated by, for example, removing the protective cap covering the discharge unit.

Included in embodiments of the invention is an actuator associated with a discharge unit that is triggered by physical contact of the device with the site of injection, typically a site on the skin surface. Typically, the injector has a discharge head containing the external openings of the ejector tubes, and is protected by a cap which removed prior to operation of the injector. On removal of the cap, the actuator is exposed and available to be triggered. Typically, the actuator is physically connected, either directly or by way of physical connections via intervening components and/or mechanisms, to the microfluidic mechanisms in such a manner that movement of the micromachined plates or valves is initiated. In some embodiments of the invention, the actuator mechanism includes an electrical component, in other embodiments the actuator events do not include an electrical component or event. In some embodiments where the operating propellant force is driven or contributed-to by a non-MEMS force, the actuator activates that propulsive process in addition to activating the movement of the MEMS plates.

In some embodiments, a pretriggering alert is included in the injection sequence. Such an alert is typically provided manually, by the user, as for example might occur in conjunction with the removal of the cap covering the discharge unit. An example of this involves an embodiment whereby the release of compressed gas provides at least some of the propulsive force. In such embodiments, the compressed gas is held in a secure compartment prior to the alert. Upon alert, the secure compartment is opened or made more ready for the final stage of release, which is triggered by the actuator.

As can be understood from the summary above, some embodiments of the injector are entirely mechanical, without any electrically-powered mechanisms or events. On the other hand, some embodiments include electrical steps within a series of mechanical processes. An electrical step, for example, may be included as part of the actuation, or as part of an alert that precedes actuation. In some embodiments, the propulsive force is provided by the MEMS components of the microfluidic apparatus, wherein propulsive force is created by one or more MEMS pumps situated between the biologically active agent reservoir, and the convergent nozzle or nozzles proximal to the emitting tubes. Power for these events, as provided by embodiments of the invention, may be stored or generated within the injector, or provided by an external source. Internal sources of power may include a battery, or electrostatic generators, or motion-driven induction. Embodiments of the invention that make use of external power include electrical contacts on the housing of the injector.

Typical embodiments of the invention are single-use, although other embodiments may be used multiple times, or have components that are reused. Single use embodiments typically include a single dosage which is delivered to completion during an injective event. Other embodiments include dosage controlling mechanisms, whereby substantially the entire and predetermined contents of the reservoir are delivered, or alternatively a fraction of the available dosage is delivered. Embodiments that are designed for single use typically are configured to be physically altered by the act of injections, such alteration serving the purpose of damaging the injector so that it cannot be reused, or such alteration may more simply cause it to be visually apparent that the device has been spent. These features which re-enforce the single use aspect of the injector serve the purpose of convenience, such that loaded and spent injectors can be quickly distinguished from each other, and such features also serve the purpose of medical safety in that they discourage the accidental contact of a spent device with a second patient.

Embodiments of the invention may vary to the degree that they are unitary, integrated, and self contained on the one hand, and on the other hand they may have modular features. Unitary, integrated, sealed injectors have the simplicity of being single use and disposable. Injectors that have modular features have their own advantages, typically advantages of flexibility either in manufacturing or in the hands of the end user.

### BRIEF DESCRIPTION OF THE DRAWINGS

FIG. 1 is a schematic representation, cutaway and partly exploded, of an embodiment of the present invention depicted in a modular format.

FIG. 2 is a pictorial representation of an embodiment of the present invention.

FIG. 3 is a sectional view along line 102 of FIG. 2 of an embodiment of the present invention with the cap removed, the discharge unit in position for application and depicting the microstream ejection of a formulation containing a biologically active agent as the device is discharged on contacting a tissue.

FIG. 4 is a side view of another embodiment of the present invention.

FIG. 5 is a cross-sectional view of an exemplary discharge unit of an embodiment of the present invention.

FIG. 6 depicts components of an exemplary micromachined valve and compression nozzles according to an embodiment of the present invention.

FIG. 7 depicts an alternative embodiment of a micromachined valve assembly.

FIG. 8 is a cross-sectional view of certain components of an exemplary micromachined valve according to an embodiment of the present invention in a closed position.

FIG. 9 is a cross-sectional view of certain components of an exemplary micromachined valve of an embodiment of the present invention in an open position.

FIG. 10 shows an exemplary actuator structure on one component of a micromachined device according to an embodiment of the present invention.

FIG. 11 is a depiction of a software-simulated profile of a microstream generated by the needleless injector.

### DETAILED DESCRIPTION OF EMBODIMENTS OF THE INVENTION

The Needleless Micro-Machined Injector

General Features of the Needleless Injector

In order to stem needle-associated disease transmission, prevent improper reuse or disposal, and alleviate anxiety caused by needle-based medicine delivery units, there is a need for a needleless medicament injector that can more safely be used and disposed of by health care workers and self-treating patients. The need for transcutaneous drug delivery units is especially acute in areas of the developing world or underserved areas, or in circumstances that involve systematic treatment of large numbers of people. In response to this need, the present technology makes use of micromachining technology in the fabrication, configuration, and administration of needleless injector of biologically active agents. Some embodiments of the injector typically single-use, other embodiments are reusable, or reusable in part. Some embodiments of the invention are modular in nature, wherein engagable subunits combine to form an integrated device. Modularity occurs in various embodiments wherein major units of the device may exist in slight variations in form that nevertheless retain points of commonality, particularly in the points where such components engage other components. In some embodiments, modular replacability presents options at the time of manufacture; in other embodiments, modularity presents options available to the end user, a health care provider, for example, in the field. As a whole, the various embodiments of the invention represent a safer, more user-friendly design, more cost effective and more easily disposed than the needle injectors currently available. The inventive drug delivery unit is appropriate for both human and veterinary uses.

Embodiments of the invention include injectors that are further characterized as being dose-or delivered-volume-specific. The dose-specified feature of the invention includes two basic variations; some embodiments are specific to a single predetermined dose, other embodiments provide the functional flexibility to deliver two or more alternative predetermined dose volumes. Variable dose delivery is useful in that it provides flexibility required for medicating patients of different weight, for example, or to provide dosing appropriate to medical need. The fine tuning of appropriate dosing may further vary in accordance with sex, age, and individual medical history and/or genetic composition. Detailed description of the hardware and mechanisms by which doses are specified or varied are provided below.

In place of needles, the present technology uses various combinations of micro-sized and/or MEMS-based components, such as valves, velocity nozzles, and ejection tubes, to create a high speed, streaming jet that penetrates a soft solid such as living tissue. Typically, embodiments of the invention comprise micromachined tubes, typically a plurality of tubes in the form of an array. The internal diameter of the individual tubes and orifices typically ranges from about 0.25μ to about 60μ, more particularly, the internal diameters range from about 0.5μ to about 40μ, and still more particularly, the internal diameters range from about 1μ to about 20μ. Orifice size and the total number of delivery tubes in a delivery unit array are factors that effect flow rate and the impact force delivered by the fluid stream. These factors may further be variables that affect the dosage delivered to the patient, either by variation in total volume delivered, or by the depth to which the drug penetrates into the treatment site on the body. Individualized and controllable dosing can be medically important for diabetic patients, for example, depending on the severity of their condition, might require 0.25 ml, 0.5 ml, or 1 ml of a particular insulin formulation.

Informative labeling of individual devices, or components of modularly-assembled devices is provided by embodiments of the invention. Labeling typically conveys medically useful information regarding the pharmaceutical agent contained within the device, such as the dosage, dates of manufacture, lot number, date of expiration, and any other information important to the end user being able to implement the device in a clinically appropriate and informed manner. Visual labeling methods or media, by way of examples, may include color coding, numerals, alphanumeric codes, logos, and universally recognized symbols. Radio frequency approaches to labeling include radio frequency tag identification (RFID). Labeling may further include any the conveyance of pertinent information by any communicating medium other than visual, including tactile and auditory stimuli.

Embodiments of the inventive needleless injector devices (transdermal, subcutaneous, intramuscular, or mucosal) injectors, i.e., devices for delivering to a tissue a dose of liquid medicament by way of a high pressure, high velocity flow stream that penetrates through the tissue and deposits the medicament subcutaneously, intramuscularly, mucosally, as for example in the mouth or other cavities in the body, or to other desired depths in the tissue being approached by the injector through the skin. The laminar flow characteristic created by the injector in some embodiments, enables delivery of a composition, such as a biologically active agent in an appropriate vehicle, more particularly a pharmaceutical drug or vaccine to a specified tissue layer with a minimal amount of pain. The injector can be configured to deliver other various charged molecules, ionic and non-ionic surfactants or other suitable suspenders; ionic or non-ionic liposomes, transfersomes (vesicles of phospholipid, surfactant, and water which are highly deformable and efficient for transdermal penetration), ethosomes (lipid vesicular carriers based on phospholipid, ethanol, and water), niosomes (non ionic surfactant vesicles), micelles; polymers, accelerants, enhancers (such as water, hydrocarbons, sulphoxides, fatty acids, esters, alcohols, amides, polyols, essential oils, terpenes, and the like), and/or solvents that would not normally be expected to penetrate through viable skin.

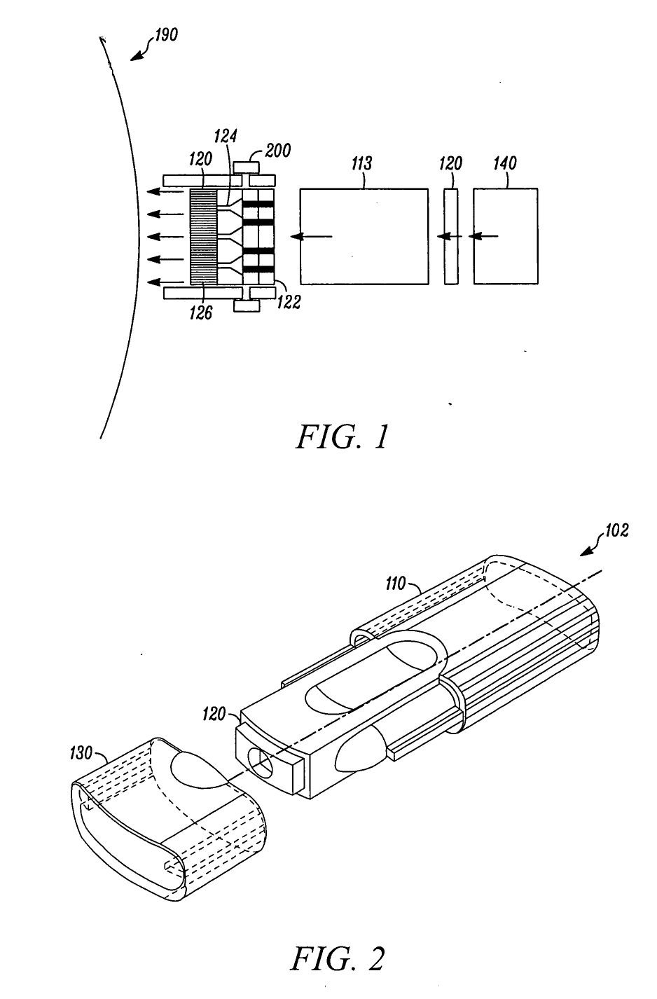

The inventive medication delivery device can be rendered in a schematic view, as shown in FIG. 1. As described herein, the needleless injector has a distal end or forward portion; from which the medication is ejected in microstreams. In terms of the microfluidic flow path, the distal end of the injector is the downstream terminus. Contrariwise, the proximal end of the device is rear portion of the device, typically it's the portion of the device held by the person performing the injection. In terms of the microfluidic flow path, the rear portion of the injector is the upstream portion, a reservoir for holding the biologically active agent may be considered the headwater. FIG. 1 depicts, starting from the distal end and moving proximally, a discharge unit 120 that houses the micromachined ejection tubes 126, typically configured as an organized array. Proximal to the discharge unit, and upstream in the microfluidic flow path is an array of micromachined compression nozzles 124. Still further proximal and upstream from the compression nozzles is a micromachined valve assembly 122 that pumps regulates and rate-limits the flow through of the liquid therapeutic composition forward into the nozzle. In some embodiments this micromachined valve assembly 122 is unpowered, and upon opening, it passively allows flowthrough of liquid under pressure. In other embodiments, the micromachined valve assembly comprises one or more MEMS pumps, which actively pull fluid through, driving it into the compression nozzles.

Extending from the most distal portion of the devise proximally to engage the MEMS device is an actuator 200 which upon appropriate distal contact, as for example, that occurs when the device is contacted to the skin of a patient, acts as a trigger for the activation of the MEMS device. Moving further proximal is a reservoir 113 for a fluid therapeutic composition. Proximal to the reservoir is force conveyer 120 that is configured between the reservoir and a propulsion unit 140. Examples of a force conveyer 120 include an elastic membrane, a diaphragm, and a plunger. Examples of a propulsion unit 140 include a chamber containing compressed gas, a chamber in which a pyrotechnic event is ignited, or a mechanical device such as a spring. These various components may vary to some degree in terms of their physical disposition with respect to being forward or rearward disposed; the distal/proximal characterization also relates to course of the microfluidic path and as well as to the functional relationships among the components. Arrows in FIG. 1 indicate the direction of force from the propulsion unit, and the direction of flow through the microfluidic system, beginning with movement of fluid from the reservoir, and culminating in the microstream ejection from the ejecting tubes.

While the therapeutic composition contained within the reservoir is typically a liquid, some embodiments of the invention are configured to contain a dry therapeutic composition that is dissolved in diluent to create the injectable formulation. Typically, dry formulations (for example, powdered, granulated, or lyophilized formulations) are useful in cases where the dry formulation has a lengthier period of stability than the dissolved or dispersed formulation. Stressors that may limit liquid formulation stability include heat, cold temperature that freezes the formulation, or changes in temperature, especially around the freezing point. Typically, embodiments that accommodate a dry formulation that is dissolved prior to injection, include a transparent window that provides a view of the liquid so that it may be visually inspected by the end user to assure that the composition is dissolved and of uniform consistency.

The disposable, embodiments of the needless injector may also include a cap that may be constructed by the various means well known in the art so as to seal the device from contaminants and maintain its contents while under pressure. In various embodiments, the cap may be securely re-attachable, or it may not be re-attachable. Re-attachment typically serves the purpose of minimizing the creation of disposables, while non-re-attachability typically serves the purpose of indicating to the end users that the device has been used, and is spent.

In some embodiments of the invention, various of these components are modular in that they are separable, engageable around standard connector portions, and may exist in varying forms, according to their use. Modular assembly of component parts may occur at varying points in the life cycle or logistical path of the device, for example, assembly may take place at the time of manufacture, or at the point of health care delivery in the field, or at any point between.

In one modular embodiment, for example, the self contained units of the front-end i.e. the plunger, medicament container, and nozzles and tubes are configured into a revolver that cycles into place, a new one for each patient. In other embodiments, the revolving configuration includes the delivery heads on the storage units with plungers built in. In another embodiment, packages of the orifices are the disposable parts, to be matched with activated pressure chambers. Examples of modular components include a dosage, a microfluidic flow creator, and an end power unit. These components may be assembled by combining a power unit onto a dosage unit that has a piston inside a cylinder. A detent is used to drop in a small CO₂ cartridge to provide propulsive force, and the result is a completely functional microstream injecting unit that can be adapted to any biologically active agent packaged into a dosage unit.

A Unitary Embodiment of the Device

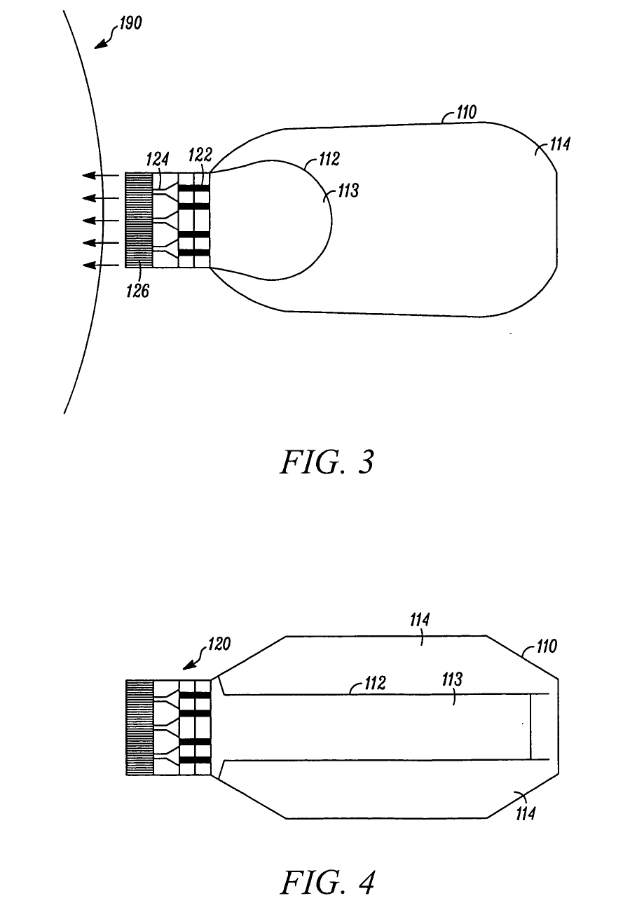

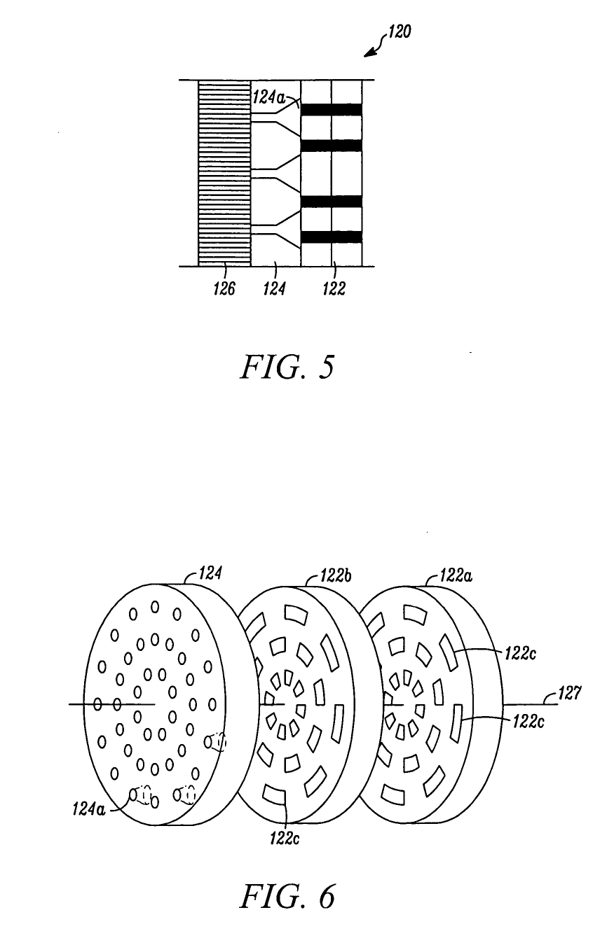

In one aspect, components of the inventive technology are embodied in a needleless hypodermic injector assembly. As illustrated in FIGS. 2 and 3, the assembly 100 includes a main injector housing 110 that includes: a reservoir chamber 114 defining a cavity, a composition chamber 113 within the reservoir, and a discharge unit 120 that contains the ejecting tubes 126. In one embodiment the reservoir is configured with a composition membrane 112 that encompasses and defines the composition chamber 113, which is adapted for storing a composition, preferably a medicament, and expelling that composition from the reservoir upon the appropriate actuation. The composition membrane may be non-porous, elastic, and may be attached to the reservoir in such a way as to effect the expulsion of the composition out of the reservoir chamber and into the discharge unit when activated.

FIG. 4 shows an alternate embodiment of the composition membrane to that shown in FIG. 3. In FIG. 3 the composition membrane has a substantially cylindrical shape, extends into the reservoir chamber 114, and is attached to a surface of the injector housing opposite the discharge device 120. Embodiments of the invention include various configurations of the composition membrane 112 that affect the desired flow characteristics as the composition is forced from the composition chamber 113 through the discharge device 120.

Returning to FIG. 3, the reservoir chamber 114 also serves as, or may further include a propulsion chamber that is adapted for housing a propulsion unit 140 that is configured to enhance the expulsion of the composition out of the reservoir and into the discharge unit. The propulsion chamber is adapted for housing a gas under pressure, such as helium, nitrogen, or carbon dioxide, that is released from the propulsion chamber when the injector device is activated. The propulsion chamber provides sufficient expulsion force to the composition membrane 112 to force the composition through a discharge unit with sufficient velocity and with appropriate flow characteristics such that the composition is transmitted through the necessary layer or layers of a patient's skin to accomplish successful injection of the particular composition into the patient.

In one embodiment a discharge unit 120 is in communication with the reservoir chamber 113 and is configured for administering a composition under pressure from the reservoir to a tissue 190. As shown in FIG. 4, the discharge unit 120 may include an array 124 of compression or velocity jets 124a and that are designed to increase the velocity of a flow from the reservoir chamber and at the same time focus the dimension of the stream into a thin, laminar or substantially laminar flow. The discharge unit further includes a micro-electro-mechanical system (MEMS) valve assembly component 122 that is in communication with both the reservoir chamber and discharge unit and is configured for regulating the release and flow of a composition from the reservoir chamber into the discharge unit. The MEMS valve component 122 may include or be functionally connected with an actuator that causes a shift in the alignment of component plates, thereby releasing the flow of the composition. Additionally, in some embodiments, the MEMS valve regulates the flow of the composition into the velocity nozzles and may include a metric actuator for delivering a predetermined amount of composition to the discharge unit. In some embodiments, the velocity nozzles, typically in an array, create a stream with almost no turbulence to reach a defined depth into the skin, while the actuator of the MEMS device and/or the velocity jets 124a will define the speed and pressure of the flow that determines the penetration depth.

The velocity jets accomplish their purpose due to their configuration of a greater opening proceeding to a smaller opening such that fluid forced through the jet reaches an ever increasing flow velocity until it passes through the smallest diameter region of the velocity jet. The geometry, location and number of the velocity jets as well as the pressure configurations of the propulsion chamber are selected as a function of many of the, dynamics of the embodiment, the density, viscosity or other flow characteristics of the fluid being discharged, the pressure and pressure drop profile of the device, the volume of fluid being discharged.

In some embodiments the assembly also includes a removable cap 130 (FIG. 2) that is configured to prevent the inadvertent triggering of the MEMS device and unintended expulsion of composition from the device as well as to protect the tube configuration from damage or contamination prior to use. The cap is removed from the discharge device prior to use of the device. In some embodiments, the cap is easily removable by a conventional mechanism such as a screw top or luer lock mechanism, and is securely replaceable. In this case, the replaceability serves to minimize medical waste clutter, as the device, itself is of a single use character. In other embodiments, the cap serves as a break seal feature inasmuch as at least some portion of the cap, albeit easily broken, is sealed to the housing or a feature associated with the discharge unit. In such embodiments, the cap is not replaceable, or at least it is not replaceable in such a manner that it appears to be in an unused state. This feature is useful in that it provides a visual or tactile information that indicates that the device is spent, and it therefore will not be confused with a used device.

FIG. 6 shows certain components of the discharge unit 120 in exploded view. Shown are two valve plates in the form of rotatable discs 122a and 122b of the MEMS device 122. As can be seen each disk 122a and 122b includes openings 122c that can be mutually aligned or misaligned as disks 122a and 122b are respectively rotated around their common axis 127. FIG. 7 shows an alternative embodiment wherein the valve plates 122a and 122b move across each other in a linear fashion (per arrows); they may be of any appropriate two-dimensional shape, not necessarily round as in the case of rotatable discs. The relative linear shift of these plates accomplishes the same objective as the rotation of the rotatable discs of FIG. 6, i.e., to adjust the openings of the respective plates into- or out of register with each other.

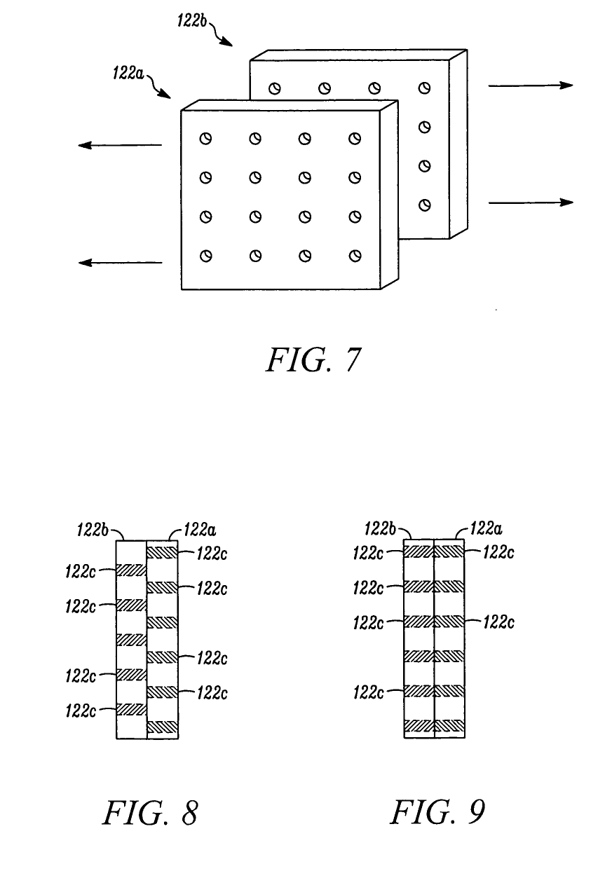

FIG. 8 shows a cross-sectional view of across the thin dimension of valve plates 122a and 122b in a misaligned condition. In this condition the micromachined valve apparatus 122 serves to prevent the flow through of the fluid formulation. FIG. 9 shows valve plates 122a and 122b in an aligned condition. In this condition the openings 122c respectively of valve plates 122a and 122b are aligned so as to allow the flow of fluid or liquid formulation through the MEMS device 122. This configuration of discs, adjacent to each other and on a common axis, with complementary holes or openings that can be rotated a few degrees in order to align or mis-align holes is a mechanism that not only controls flow of therapeutic composition in an on/off sense, but can be exploited to control dose delivery, as is described below, in the section “dosage control mechanisms”.

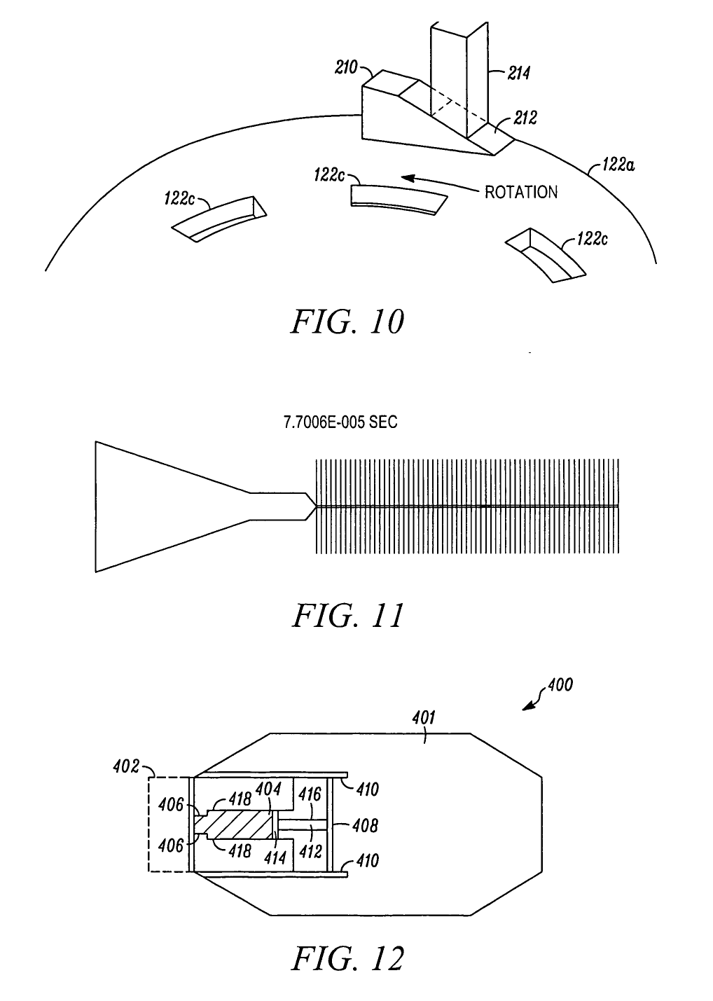

FIG. 10 illustrates an exemplary actuator mechanism 200 for rotatable disk embodiment of valve plate 122a or 122b into an aligned position to allow flow of fluid through MEMS device 122. The mechanism includes a raised element 210 having a sloped surface 212. Against sloped surface 212 is aligned a force rod 214 that transfers force from the impact of the discharge unit 120 against flesh-190. The force of the impact is transferred down the force rod 214 and against sloped surface 212 causing disk 122a to rotate counter-clockwise. Two, four or another number of raised element-force rod combinations can be employed to rotate disk 122a when the discharge unit is forced against a surface such as the surface of the body of an animal or human 190. It will be understood that the force rods or some additional elements will extend, to the end of, or beyond the end of, discharge unit 120 so that force is transferred from the impact of the discharge unit 120 against the surface to the sloped surface 212. Additionally, other suitable actuator assemblies may be employed. The actuator mechanism may be employed to rotate either of disk 122a or disk 122b.

In one exemplary embodiment of the present invention, the reservoir includes two sheets of thermoplastic material, wherein each of the two thermoplastic sheets is comprised of an expanded central portion and an expanded administration neck portion. The expanded central portions of the two sheets of thermoplastic material are opposite and coextensive to each other and are sealed together face-to-face, and configured to form a reservoir for storing a composition, such as a medicament, under pressure. The expanded administration neck portions extend from the expanded central portions to a first end of each sheet, and are opposite and coextensive to each other, and configured to form an administration housing extending from the reservoir and connected to the discharge unit.

Additionally, a composition-enclosing membrane may be included that is in communication with a distal portion of the reservoir chamber and is configured for storing a composition and expelling that composition into the discharge unit upon appropriate activation of the device. A pressure chamber in communication with the reservoir chamber may also be included. Ideally, it will be located proximal to the composition membrane of the device and is configured for housing a propulsion device that may simply be a gas under pressure that may be released upon appropriate activation of the device. In another embodiment the composition chamber and pressure chamber are formed of the reservoir and are the same entity.

The disposable injector may be formed of two sheets of thermoplastic material that are sealed together face-to-face with an inter-lying composition membrane, prior to formation of the expanded central portions of the two sheets. The central portions of the two sheets and the administration neck portion extending there from are expanded to form the reservoir (encasing the composition membrane) and propulsion chamber, as well as the discharge unit. After the peripheral portions are sealed together, the reservoir may be formed by injecting compressed gas between the appropriate portions of the two sheets or by drawing apart the appropriate portions of the two sheets using a vacuum, until those portions expand to fill a cavity of a mold.

Unitary embodiments of the invention (in contrast to devices that may be assembled from component modules, as described above) may be labeled by visual or radio frequency approaches. Such labeling may be present on the item itself, and/or alternatively, on wrappers that cover the devise, or on tags connected to the device, as has been described above.

Micro-Electro Mechanical System Components

In some embodiments of the invention, the discharge unit further includes a micro-electro-mechanical system (MEMS) valve component for regulating and directing the composition from the reservoir to the velocity nozzles and a micromachined tube array assembly. MEMS devices can be fabricated in many various ways that are well known in the art; typically they are fabricated using integrated-circuit (IC) and silicon micromachining technology, have overall dimensions on the order of a millimeter, and can have component sizes (e.g. housing, gears, rotors, linkages, levers) on the order of microns.

When included in the device, the MEMS valve assembly may be comprised of two complementary plates that one or both of which may respond to an actuation event. For instance, a release lever may need to be depressed before actuation of one or both of the spindle plates can occur. Accordingly, in response to an activating pressure transferred to the MEMS valve, the spindle plate(s) move(s) from a closed to an open position effecting the expulsion of the composition from the reservoir, through the MEMS valve, into the compression nozzles, and out through the micromachined tube array. Ideally, once activated the MEMS valve may be configured to lock into place so as to prevent the device from being reused, of course the MEMS assembly can also be configured to allow reuse.

In one particular embodiment, the first and second plates are in a housing that is formed along with the two plates using MEMS technology, wherein either the first or second plate moves from a closed position to an open position, in relation to the other plate which does not move, when pressure is applied to a lever mechanism. In a different embodiment, a gear and rack mechanism is included along with a rotating member, wherein the gear, rack, and rotating member are formed along with the plates using MEMS technology, and they are incorporated to actuate movement of either both plates, or one plate in relation to another, in a circular rotation from a closed to an open position. In all these instances, the cutout holes are either unshaped or shaped openings that are sized so that when the two plates and are in the open position the orifices formed by the aligned cutouts allow for an unrestricted flow. However, it will be understood that the MEMS valve can be triggered by any number of structures designed to open the valve when a pressure is applied, or by other triggering events, as may be useful for the particular embodiment.

Micromachined Injector Tube Array

In various embodiments, the array of hollow velocity nozzles comprise an array of tubes that can be fabricated in micrometer dimensions to have a range of specific geometries by the micro-electric etching of silicon, metal, polymers, glass substrates, and the like. In the context of the microelectronics revolution, highly precise and scalable fabrication methods have been developed that allow for the rapid, mass production of micro-nozzle arrays in so-called MEMS Fabs. Micromachined components such as ejecting tubes, velocity nozzle arrays, and valvles can be fabricated en masse, as is well known in the microelectronic fabrication arts, by use of fabrication masters to produce molds from which a multiplicity of arrays can be fabricated.

Accordingly, silicon, metal, polymer, and/or glass or the like, can be used as substrates from which fabrication masters can be etched, from these masters, micromolds can be fabricated for the mass production of micro-nozzle arrays. The size, shape, dimensions, and configuration of the arrays can be precisely engineered to produce the precise flow-through characteristics desired of the velocity nozzles by many well known techniques, such as by reactive ion etching. In a particular embodiment the velocity nozzles are configured to deliver a composition or drug from the reservoir to the micro-machined tube array.

The certain embodiments, the combination of velocity nozzles with the micromachined tube array creates a thin stream jet through which a composition flows with a laminar flow characteristic. Due to the dynamics of the system a composition and/or drug(s) therein can be administered in a pain free way with minimal risk of disease transmission through misuse or inadvertent care. Because of the higher pressure achieved, and the focused laminar flow, the micromachined tube injector allows for the effective delivery of a medicament through the stratum corneum to the deeper layers of the epidermis without causing long lasting damage and with undetectable or minimal triggering of pain receptors.

Dosage Control Mechanisms

For the appropriate functioning of the MEMS-based needleless injector, embodiments of the invention include various mechanisms for controlling the dosage of therapeutic composition delivered. Some embodiments have a dosage that is fixed by the total amount of dosage contained in the reservoir, the injector configured to deliver substantially all of the content. Other embodiments include features and mechanisms that provide variable dosages that can be delivered by a single unit with a fixed volume in the reservoir. Such embodiments include, for example, methods and mechanisms controlling the volume displaced by movement of a piston, controlling the size and configuration or distribution of openings created by the common alignment of the openings in the rotatable discs, and controlling the time interval during which the plates are aligned to create the contiguous flowpath openings.

In one embodiment, the movement of piston mechanism being driven from its distal end is stopped in its forward movement by a barrier. The volume of the fluid displaced by the forward movement of the piston is a product of the diameter of the piston and the distance of forward movement. In another embodiment, the dosage is controlled by the controlling the size of the common area defined by the coincidence of the openings of the two rotatable discs. In another embodiment, the dosage is controlled by controlling the duration of the time interval when the rotatable discs coincide to create a contiguous flow path. In one embodiment of this type, the time interval is controlled by a self closing feature of the discs, whereby fluid flow or piston movement is coupled to a torque on one of the discs such that the disc moves to a closed position with respect to the open area defined by coincidence of the hole area on the complementary discs.

One typical embodiment of the invention includes a liquid or gel composition for delivery to a subject, some embodiments include a biologically active composition in a powdered, granular, lyophilized, or otherwise dry state and an amount of liquid diluent. The dry and liquid components, in these embodiments, are joined together to effect dissolving of the dry agent in the liquid at a time prior to injection, and the liquid composition is then injected in the manner as has been described above.

Iontophoretic Agent Delivery Embodiments

Micromachined tube based injector can be constructed in such a way that the discharge unit acts as an electrode. In one embodiment, the micromachined tubes within the array act as electrodes and maybe in communication with an electrode plate at the distal end of the device that comes into contact with a subject's tissue before administration, or the micromachined tubes themselves may be designed to come into contact with a subject's skin without the use of an electrode plate. Additionally, the micromachined tube electrodes may further comprise or include conductors and are operatively connected, via the appropriate connectors, to a voltage source that may either be internal or external to the device and may generate a direct or alternating current. In this embodiment the injector additionally acts as an iontophoresis device further driving the medicament deeper into the tissue. In this instance, the micromachined tubes and/or electrode plate act as driving electrodes and will be of the same charge as the medicament to be delivered, or for non-polar molecules the micromachined tube electrodes will be the same charge as a suspension in which the non-polar molecules are suspended. Further, an additional grounding electrode may be used and placed elsewhere on the subject's body to complete a circuit.

In a related embodiment the micromachined tube array comprise electrodes that may be differentially charged. In this embodiment a suitably configured circuit board may be included with appropriate MEMS constructed conductors so as to direct appropriate and opposite charges to various combinations of the micromachined tube electrodes. In this embodiment the injector device will be configured such that the micromachined tube electrodes make contact with the tissue, and are in operational communication, via appropriate connectors, to a suitable voltage source. Once in contact with the tissue the voltage source may be activated so as to deliver a pulsed energy field between oppositely charge electrodes and the injector may be actuated to deliver the medicament either concurrently, before, or after the delivery of the electroporating pulses. In another embodiment the injector device is configured to deliver iontophoresis, hydrophoresis, and/or electroporation or any combination thereof.

Nanotube-Based Embodiments

Some embodiments of the invention provide nanotube-based ejecting tubes configured into the discharge unit, receiving microfluidic flow from upstream micromachined convergent jets. These nanotube-based tubes are typically formed of a cylindrical arrangement of parallel individual nanotubes. The void diameter of such tubes ranges from 10 nm to more than 100 nm, a range that is between about 5% to about 50% of the lower limit of the micromachined tubes described elsewhere herein. The much small diameter of the nanotube structures allows the creation of nanostream jets which are particularly useful in intracellular injection procedures. Design ramifications of nanotube structures include generally smaller volumes of injectate, higher numbers of ejecting tubes, and a requirement for higher propulsive force in order to move fluid.

Needleless Injector Partnered with a Specific Therapeutic Formulation

Some embodiments of the invention are manufactured in an “open” way, such that they may be filled with any of a variety of therapeutic formulations, at varying dosages, by various pharmaceutical companies or any of a variety of vendors or distributors of pharmaceutical products. Other embodiments of the invention, however, include needleless injectors that are designed for loading with a specific pharmaceutical agent or agents, as manufactured, for example, by a specific pharmaceutical company. The specificity of the intended use by such composition-dedicated injectors allows for custom manufacture to the specifications of the pharmaceutical company, including, for example, custom labeling on the housing of the injector that provides useful information to the end users, or other variations of hardware or fittings as may be appropriate for the context of the intended use. Typically, these embodiments, as delivered to the end user, are single-use devices, fully integrated and sealed. Further, such conjoining or partnering of the injector with the pharmaceutical to be delivered, encourages the development of formulations that may be particularly well adapted for administration by the needleless injector.

Treatment Kits Comprising Multiple Injectors

Various embodiments of the invention include treatment kits that contain a plurality of individual needleless injectors, where the injectors are organized in a manner appropriate for a clinical or emergency situation. The aggregate of injectors may include duplicates, identical in terms of the included therapeutic composition and dosing, or they may include injectors of varying dose level of a single therapeutic composition, or they may include injectors loaded with different therapeutic compositions, or any of these various combinations. As provided by the invention, the individual devices in such kits containing multiple devices are labeled in ways that make them readily identifiable and distinguishable from each other. Such labeling may be provided by any method known to be effective, for example by any of written language, by numbers, by universally understood pictographs, by color coding of background space or of information conveying symbols, or by any combination of these communicative methods. Labeling, per various embodiments of the invention, may include markings and color coding integrated into or on the surface of the injector, on the housing, for example, or on any other appropriate surface. In other embodiments, such markings and color coding may be located on wrappers around the individual devices, or on tags connected to the individual devices. In addition to the visual labeling methods just described, in some embodiments, labeling is provided by radio frequency identification (RFID) methods, which are able to provide a full complement of information about a labeled device to a user at any point in the life cycle or logistical stage of the device from the point of manufacture to end use.

By way of example, on a battlefield, military medical personnel may anticipate the need for countermeasures to various chemical or biological agents, in addition to standard anesthetics or narcotics. Similarly, in geographical areas that have been hit by a natural or man-made disaster, a particularly focused array of medications may be appropriate. Such a selection of treatment-loaded needleless injectors may be predetermined at the time of manufacturing; in other words, the full assembly of the kit would take place by a manufacturer prior to sale. In other embodiments, the kit and its component injectors may be assembled after sale of component injectors, by intermediate users or health care providers in the field. Typically, kit-type embodiments of the invention have a premium on the physical security and compactness of the kit, and quick accessibility to a range of possibly needed treatments; less of a premium would be placed on complete utilization of all included treatment-loaded injectors.

### EXAMPLES

### Example 1

### Microstream Injection Studies with Gel Models of Tissue

Embodiments of the invention are tested in polyacrylamide gel systems that stand in for skin and underlying connective tissue, fat, and muscle. Examples of such systems are described by Schramm-Baxter, Katrencik, and Mitragotri (“Jet injection into polyacrylamide gels: investigation of jet injection mechanics”, J. Biomechanics 37 (2004) 1181-1188), Schramm-Baxter and “Needle-free injections: dependence of jet penetration and dispersion in the skin on jet power”, J. Controlled Release 97 (2004) 527-535), and by Shergold, Fleck, and King (“The penetration of a soft solid by a liquid jet, with application to the administration of a needle-free injection, J. Biomechanics, in press 2005, available to online subscribers). In addition to the use of homogeneous gels, biphasic gels are tested, with a tough outer layer, to mimic the stratum corneum, over a softer layer, to mimic the underlying epidermis, dermis, connective tissue, fat, and muscle. By using these systems, the depth and diffusion pattern of injectate can be photographed and analysed. Using such data allows tuning of operational parameters such as force applied, the time course of force application, and the dimensions of all the components of the microfluidic path.

### Example 2

### Computer Simulations of Microstream Injections

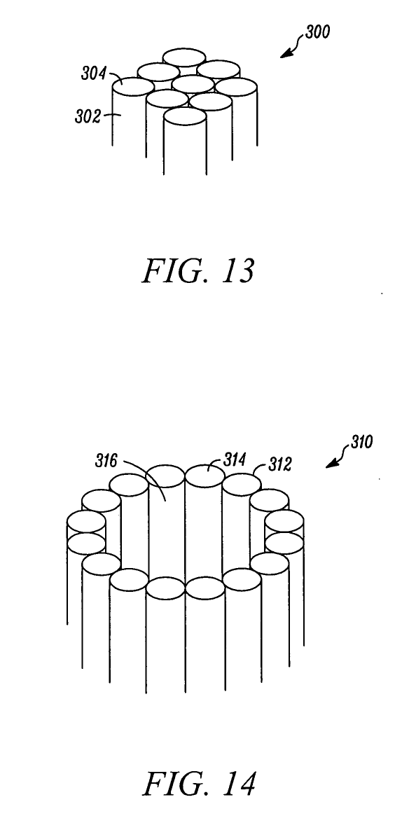

Computer simulation provides another approach to designing and testing the injector. The inventor has made use of ASE+, a program designed by NASA Marshall Flight Center and the CFD Research Corporation (Huntsville, Ala.), now owned by ESI Corportation (headquarters in Portland Oreg.). As a test model, a nozzle with an upstream width of 500μ, narrowing to a 60μ funnel end with a convergent angle of 60 degrees, emptying into an ejecting tube of 20μ diameter was tested. According to the simulation, an ejection velocity of 60 m/sec is supported by a pressure upstream of the ejecting tube of 3.9 MPa, a velocity of 80 m/sec is achieved with 6.8 MPa, and a 100 m/sec velocity is supported by a pressure of 10.6 MPA. This amount of pressure compares favorably with conventional pressure systems, a CO₂ gas cartridge, for example, is filled to a presure of about 7 MPa. In addition to the data supporting the feasibility of attaining appropriate ejection velocities, the simulation shows that the jet emerges in a single, unbroken, coherent microstream, as shown in FIG. 11.

### Example 3

### In Vitro Transdermal Microstream Injection Studies

Rates of transdermal transport are determined by contacting human cadaver epidermis with the injector of the invention and activating the device. Skin is obtained from the abdomen or back of human cadavers (with approval of the appropriate Institutional Review Boards). Epidermis is isolated by incubating in 60° C. water for 2 min and gently removing epidermis tissue. Because the primary barrier to transdermal transport is the stratum corneum (the upper 10-15 μm of the epidermis), the use of epidermis rather than full-thickness skin is a well established model for transdermal drug delivery.

Isolated epidermis is mounted in a Franz diffusion chamber at 37° C. and is bathed in the receiver compartment (lower, viable epidermis side) with PBS, and in the donor compartment (upper, stratum corneum side) with 1 mM calcein (Sigma), 100 units/ml insulin (Humulin-R, Eli Lilly), 80 uM Texas red-labeled BSA (Molecular Probes), or polystyrene latex nanospheres at concentrations of 2.5×10¹⁴ nanospheres per ml (25-nm radius) and 2.4×10¹⁴ nanospheres per ml (50-nm radius) (Polysciences, Warrington, Pa.). To prevent insulin aggregation, 10 mM n-octyl 3-D-glucopyranoside (Sigma) is added to donor and receiver solutions when insulin is present.

Skin permeability is determined by measuring solute concentrations in the receiver compartment over time by calibrated spectrofluorimetry (calcein, BSA, and nanospheres) or radioimmunoassay (insulin; Linco Research, St. Charles, Mo.). Skin is temporarily removed from the diffusion chamber and placed on a supported surface whenever the injector is contacted with the sample. Epidermis samples are contacted with an injector that is then activated to release a composition into the sample and then are chemically fixed with formalin and freeze dried after dehydration using graded ethanol substitution. Gold-coated samples are then examined by scanning electron microscopy.

### Example 4

### In Vivo Microinjection Studies

The ability of the needleless injector to propel compounds into skin is assessed in diabetic, hairless rats by using an approved protocol. Hairless rats (healthy, male, adult, 300-400 g) are injected i.v. with 100 mg/kg streptozotocin (Sigma) in a volume of 700 μl. Over the next day, diabetes develops because of destruction of pancreatic islet cells by streptozotocin. Before microinjection studies, rats are anesthetized by i.p. injection of urethane (Sigma) and are verified to have successful induction of diabetes by verifying blood glucose levels of 350-500 mg/dl.

An embodiment of the inventive needless injector is then filled with either PBS or 100 Units/ml insulin (Humulin-R) and contacted with the dorsal skin to inject fluid into the skin. Blood is collected periodically by tail vein laceration and assayed for glucose concentration (Accu-chek Compact blood glucose meter, Roche Diagnostics).

### Example 5

### In Vivo Intracellular Microstream Injection Studies

The ability of an embodiment of the inventive needleless injector to effectively inject a biologically active agent into a living cell and have the cell survive the procedure is demonstrated in cell culture systems. As an example, DU145 cells (American Type Culture Collection) are cultured as monolayers in a 100×15 mm sterile polystyrene dishes (Fisher Scientific) by using RPMI 1640 growth medium (Mediatech, Herndon, Va.) and supplemented with: 10% (vol/vol) FBS (Mediatech). Cell cultures are incubated at 37° C. and 95% relative humidity in a 5% C02/95% air atmosphere until they form a confluent monolayer.

After decanting the cell growth medium, 10˜M calcein (Molecular Probes) in PBS (Sigma) is added to the Petri dish. An embodiment of the inventive microstream injector fitted into a micromanipulating apparatus is contacted with minimal force to a portion of the monolayer, activated, and removed after 5 seconds. Two minutes later, the calcein solution is decanted and the cells are washed three times with PBS. Finally, a solution of 10 μg/ml propidium iodide (Molecular Probes) is added to stain nonviable cells. Intracellular uptake of calcein (green fluorescence) and loss of viability are indicated by propidium iodide staining (red fluorescence) and are determined by imaging monolayers by using fluorescent microscopy (Olympus).

### EQUIVALENTS OF THE INVENTION

While particular embodiments of the invention, examples, and variations thereof have been described in detail, other modifications and methods of using the disclosed therapeutic combinations will be apparent to those of skill in the art. The accompanying drawings are incorporated in and constitute a part of this specification; they illustrate embodiments of the technology, and together with the description, serve to explain the principles of the invention. Various terms have been used in the description to convey an understanding of the invention; it will be understood that the meaning of these various terms extends to common linguistic or grammatical variations or forms thereof. Although the description offers scientific theory and interpretation of data, it should be understood that such theory and interpretation do not bind or limit the claims. It should be understood that various applications, modifications, and substitutions may be made of equivalents described and depicted in the specification without departing from the spirit of the invention or the scope of the claims. Further, it should be understood that the invention is not limited to the embodiments that have been set forth for purposes of exemplification, but is to be defined only by a fair reading of the appended claims, including the full range of equivalency to which each claim element thereof is entitled.

What is claimed is:

1. A needleless injector comprising a microfluid transport assembly, the assembly comprising one or more microstream ejecting tubes that range in diameter from about 0.25μ to about 60μ, the assembly configured to propel an injecting microstream containing a biologically active agent into an injection site from each tube.

2. The injector of claim 1, wherein the microfluid transport assembly comprises one or more microstream ejecting tubes that range particularly in diameter from about 0.5μ to about 40μ.

3. The injector of claim 1, wherein the microfluid transport assembly comprises one or more microstream ejecting tubes that range more particularly in diameter from about 1μ to about 20μ.

4. The injector of claim 1, wherein the microfluid transport assembly further comprises:
one or more convergent nozzles upstream of the microstream ejecting tubes,
a valve apparatus upstream of the one of more convergent nozzles, and
a reservoir for holding the biologically active agent, upstream of the valve apparatus,
wherein the assembly forms a microfluidic flow path originating in the reservoir, and terminating at the external opening of each tube.

5. The injector of claim 4, wherein the valve apparatus comprises at least two plates adjacent to each other across the flow path, each plate comprising complementary holes, said complementary holes coincinding to create a contiguous microfluidic flow path when the plates are aligned in an open position.

6. The injector of claim 4, wherein the at least two plates of the valve apparatus are in the form of rotatable discs, and wherein rotation of one the discs with respect to the other adjusts the alignment of the respective sets of openings to regulate microfluidic flow.

7. The injector of claim 4, wherein the at least two plates of the valve apparatus are in the form of non-rotatable plates, and wherein a linear shifting of one plate with respect to the other adjusts the alignment of the respective sets of openings to regulate microfluidic flow.

8. The injector of claim 4, wherein the valve apparatus comprises at least one MEMS pump, said pump interposed in the microfluidic flow path between the convergent nozzles and the reservoir, wherein the at least one pump provides a propulsive force conveying the biologically active agent from the reservoir toward the microstream ejecting tubes.

9. The injector of claim 1, further comprising:
a discharge unit configured to accommodate the external openings of the ejecting tubes and to make contact with the injection site,
an actuator associated with the discharge unit, the actuator configured to contact the injection site before an operation of the injector and to be in functional connection with the microfluid transport assembly, and to respond to contact with the injection site, the response activating the operation of microfluid transport assembly.

10. The injector of claim 9, wherein the functional connection between the actuator and the microfluid transport assembly is by physical coupling, without an electrical component.

11. The injector of claim 9, wherein the functional connection between the actuator and the microfluid transport assembly includes an electrically-operated component.

12. The injector of claim 1, further comprising an electrostatic generator apparatus, the generator configured to power the operation of one or more components of the injector that require power to operate.

13. The injector of claim 1 further comprising an external electrical contact for receiving power for the operation of the injector.

14. The injector of claim 4, wherein the reservoir holds a predetermined amount of the biologically active agent for injection, the agent included in a formulation selected from the group of a liquid formulation or a gel formulation.

15. The injector of claim 4 wherein the reservoir separately holds a predetermined amount of dry biologically active agent and a predetermined amount of a liquid diluent, the reservoir configured to combine the agent and the diluent prior to initiating the microfluidic flow.

16. The injector of claim 1 further comprising a propulsive module, the module configured to provide the force to propel the one or more injectable microstreams from the one or more ejecting tubes, the propulsive force being provided by an event within the module selected from the group consisting of the expansion of compressed gas, a pyrotechnic event, and the release of a mechanical spring.

17. The injector of claim 16, further comprising a force conveyer interposed between the propulsive module and the reservoir such that the biologically active agent is moved through the microfluid transport assembly and toward the ejecting tubes, the force conveyer selected from the group consisting of a membrane, a diaphragm, and a piston.

18. The injector of claim 1, further comprising labeling with medically useful information, the medium being one or more forms selected from the group consisting of visual information, auditory information, tactile information, and radio frequency identification.

19. The injector of claim 1, wherein the injector is a single-use injector.

20. The single-use injector of claim 17, wherein the injector is configured to be physically altered by a single usage, such alteration being apparent to a user on visual inspection of the injector and indicating that the injector has been used.

21. The injector of claim 1, wherein the injector is configured to deliver a variable dosage of the biologically active agent.

22. A kit comprising a plurality of individual needless injectors as in claim 1, each of the individual injectors labeled with clinically useful information, the medium being one or more forms selected from the group consisting of visual information, auditory information, tactile information, and radio frequency identification.

23. A needleless injector comprising a microfluid transport assembly, the assembly comprising:
one or more microstream ejecting tubes,
a discharge unit housing the ejecting tubes,
one or more convergent nozzles upstream of the ejecting tubes,
a valve apparatus upstream of the convergent nozzles,
a reservoir for holding a biologically active agent upstream of the valve apparatus, and
a propulsion unit for propelling fluid from the reservoir to the ejecting tubes.

24. The injector of claim 23, wherein one or more of the elements selected from the group consisting of the discharge unit, the reservoir, and the propulsion unit are modularly replaceable.
